(19) United States
(12) Patent Application Publication
(10) Pub. No.: US 2010/0240926 A1
(43) Pub. Date: Sep. 23, 2010

(54) PROCESS FOR PREPARING (R)-N-BENZYL-2-(BENYLOXYCARBONYLAMINO)-3-METHOXYPROPIONAMIDE

(75) Inventors: Gordana VRBANEC, Zagreb (HR); Miroslava HARCA, Krizevci (HR); Ana SUCEC, Velika Gorica (HR); Damir SAHNIC, Zagreb (HR); Amir AVDAGIC, Zagreb (HR)

Correspondence Address: MERCHANT & GOULD PC, P.O. BOX 2903, MINNEAPOLIS, MN, 55402-0903, US

(73) Assignee: PLIVA HRVATSKA D.O.O., Zagreb (HR)

(21) Appl. No.: 12/726,767

(22) Filed: Mar. 18, 2010

Related U.S. Application Data
(60) Provisional application No. 61/161,181, filed on Mar. 18, 2009.
(60) Provisional application No. 61/172,903, filed on Apr. 27, 2009.

(51) Int. Cl. C07C 231/12

(52) U.S. Cl. 564/155

(57) ABSTRACT

(R)—N-benzyl-2-(benzyloxycarbonylamino)-3-methoxypropionamide is an intermediate useful for preparing lacosamide. It can be prepared, for example, by combining (R)—N-benzyl-2-(benzyloxycarbonylamino)-3-hydroxypropionamide with dimethylsulfate, followed by mixing with an alkali or alkaline earth metal hydroxide at a temperature of about 25° C. to about −15° C.

### CROSS-REFERENCE TO RELATED APPLICATIONS

This application claims the benefit of U.S. Provisional Patent Application Ser. Nos. 61/161,181, filed Mar. 18, 2009, and 61/172,903, filed Apr. 27, 2009, which are incorporated herein by reference.

### FIELD OF INVENTION

The present invention relates to a process for preparing (R)—N-benzyl-2-(benzyloxycarbonylamino)-3-methoxypropionamide, an intermediate of Lacosamide.

### BACKGROUND OF THE INVENTION

Lacosamide, (R)-2-acetamido-N-benzyl-3-methoxy-propionamide, has the following formula:

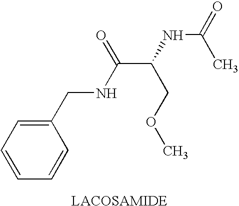

It is a drug that has been used in the treatment of epilepsy.

Lacosamide is marketed under the trade name Vimpat® by UCB. It was approved by the FDA as an adjunctive therapy for partial-onset seizures in October 2008.

Lacosamide and its preparation are disclosed in U.S. Pat. No. 6,048,899, a continuation in part of No. 5,773,475 (hereinafter, “U.S. '899”). There, Lacosamide is prepared from (R)—N-benzyl-2-(benzyloxycarbonylamino)-3-methoxypropionamide of formula II, which is prepared by O-methylation of (R)—N-benzyl-2-(benzyloxycarbonylamino)-3-hydroxypropionamide of formula I. In U.S. '899 the O-methylation requires a large excess of methyl iodide and silver oxide, an expensive reagent, in acetonitrile. The U.S. '899 process can be illustrated by the following scheme:

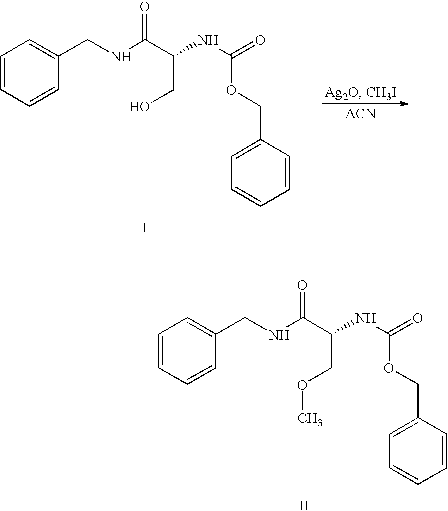

U.S. '899 reports that the above reaction takes about 3 to 4 days at room temperature, and then the Formula II product is recovered and purified, prior to its conversion to lacosamide. The recovery in U.S. '899 requires filtration of salts such as Ag₂O and AgI. The purification in U.S. '899 requires column chromatography, which is a time consuming operation and which is not desirable for industrial scale synthesis.

US 2008/0027137 (“U.S. '137”) teaches that the process using silver (I) oxide and methyliodide is impractical and expensive and results in partial racemization which reduces the product yield and enantiomeric purity. In addition, it is mentioned that the removal of the S-enantiomer that was introduced by the racemization is extremely difficult.

Thus, there is a need in the art for an improved process for preparing (R)—N-benzyl-2-(benzyloxycarbonylamino)-3-methoxypropionamide which is also suitable for industrial scale.

### SUMMARY OF THE INVENTION

According to one embodiment, the invention comprises a process for preparing (R)—N-benzyl-2-(benzyloxycarbonylamino)-3-methoxypropionamide of formula II:

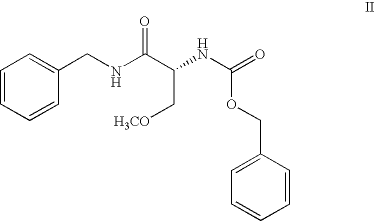

comprising (a) combining (R)—N-benzyl-2-(benzyloxycarbonylamino)-3-hydroxypropionamide of formula I,

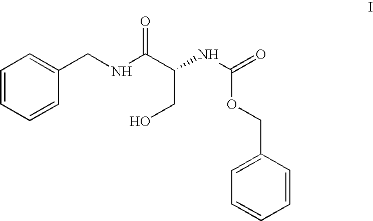

and dimethylsulfate, and then (b) adding an alkali or alkaline earth metal hydroxide at a temperature of about 25° C. to about −15° C.; and optionally isolating (R)—N-benzyl-2-(benzyloxycarbonylamino)-3-methoxypropionamide from the reaction mixture.

In another embodiment, the invention encompasses a process for preparing Lacosamide of the following formula

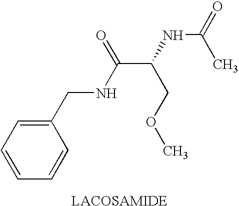

by a process comprising preparing (R)—N-benzyl-2-(benzyloxycarbonylamino)-3-methoxypropionamide of formula II, according to the above process and converting it to Lacosamide.

### BRIEF DESCRIPTION OF THE FIGURES

FIG. 1 shows a PXRD pattern of crystalline (R)—N-benzyl-2-(benzyloxycarbonylamino)-3-hydroxypropionamide.

FIG. 2 shows a DSC thermogram of crystalline (R)—N-benzyl-2-(benzyloxycarbonylamino)-3-hydroxypropionamide.

FIG. 3 shows a PXRD pattern of crystalline form I of (R)—N-benzyl-2-(benzyloxycarbonylamino)-3-methoxypropionamide.

FIG. 4 shows a DSC thermogram of crystalline form I of (R)—N-benzyl-2-(benzyloxycarbonylamino)-3-methoxypropionamide.

FIG. 5 shows a PXRD pattern of crystalline form II of (R)—N-benzyl-2-(benzyloxycarbonylamino)-3-methoxypropionamide.

FIG. 6 shows a DSC thermogram of crystalline form II of (R)—N-benzyl-2-(benzyloxycarbonylamino)-3-methoxypropionamide.

### DETAILED DESCRIPTION OF THE INVENTION

The preparation of (R)—N-benzyl-2-(benzyloxycarbonylamino)-3-methoxypropionamide of formula II can be achieved by O-methylation of (R)—N-benzyl-2-(benzyloxycarbonylamino)-3-hydroxypropionamide of formula I. However, (R)—N-benzyl-2-(benzyloxycarbonylamino)-3-hydroxypropionamide of formula I can also undergo an N-methylation side reaction providing an impurity which is ((R)-benzyl 1-(benzylamino)-3-methoxy-1-oxopropan-2-yl(methyl)carbamate (“N-Me II”), as described by the following scheme:

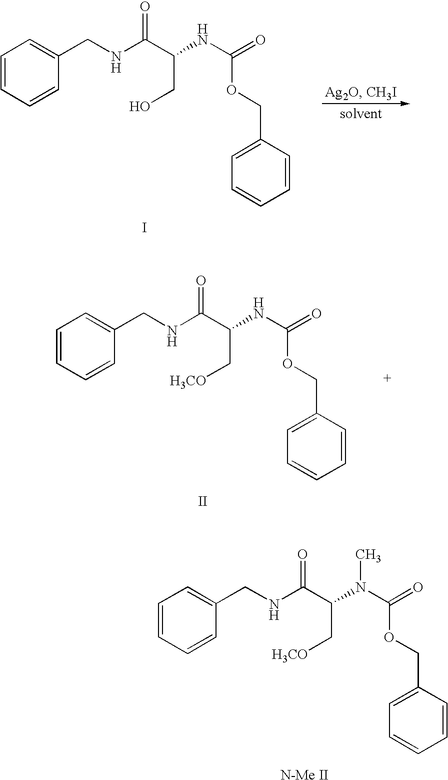

If the N-Me II impurity is not removed, it will react under the conditions used to convert the formula II compound to Lacosamide. This side reaction can form another impurity, N-methylated Lacosamide (“N-Me Lacosamide”) of the following formula:

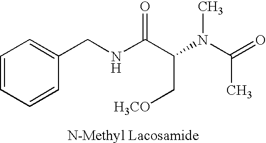

As shown in comparative example 4 herein, both N-Me II and N-Me Lacosamide have been found to be formed in the process taught by U.S. '899. As can be seen, there is considerable structural similarity between the above N-methylated impurities, the synthetic intermediate, (R)—N-benzyl-2-(benzyloxycarbonylamino)-3-methoxypropionamide of formula II and the product, Lacosamide. These two impurities are structurally similar to Lacosamide, and can complicate the task of purifying Lacosamide, while maintaining a commercially acceptable yield.

Thus, the process reported in U.S. '899 suffers from at least the following drawbacks:

Silver oxide and acetonitrile are very expensive.

The obtained product of formula II has to be recovered and purified prior to its conversion to Lacosamide, due to the presence of salts such as Ag₂O and AgI.

The recovery from the reaction mixture contains a complex composition of colloidal salts and impurities which is very difficult to handle and potentially would significantly decrease the yield; see comparative example 4 infra.

The purification of (R)—N-benzyl-2-(benzyloxycarbonylamino)-3-methoxypropionamide of formula II and Lacosamide from the N-methylated impurities via chromatography and crystallizations are not successful.

The reaction time is very long and thus is disadvantageous for industrial manufacture.

The conversion of the synthetic intermediate (R)—N-benzyl-2-(benzyloxycarbonylamino)-3-methoxypropionamide containing the N-Me II impurity to Lacosamide provides impure lacosamide.

The present invention offers an improved method for methylating (R)—N-benzyl-2-(benzyloxycarbonylamino)-3-hydroxypropionamide of Formula I in a selective manner. The process provides (R)—N-benzyl-2-(benzyloxycarbonylamino)-3-methoxypropionamide of formula II in a higher yield and purity, especially purity with respect to the N-Me II impurity, without the need to purify the product prior to its conversion to Lacosamide. Thus, when converting (R)—N-benzyl-2-(benzyloxycarbonylamino)-3-methoxypropionamide of formula II to Lacosamide, the obtained Lacosamide is provided free of the N-methylated by product, even without purification.

The preferred process of the present invention can be illustrated by the following scheme:

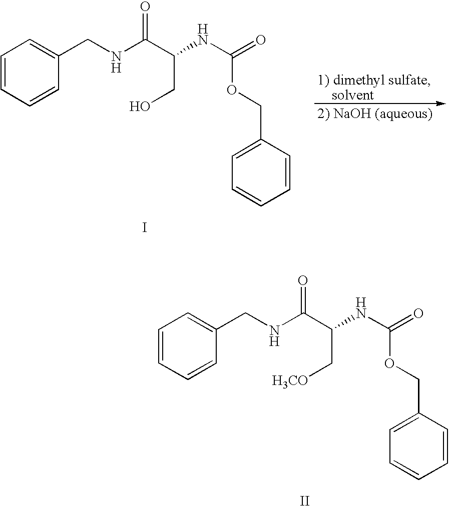

The process comprises (a) combining (R)—N-benzyl-2-(benzyloxycarbonylamino)-3-hydroxypropionamide of formula I:

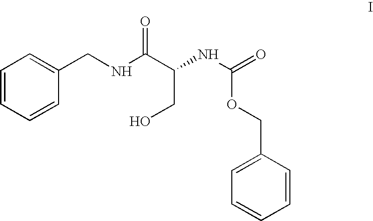

and dimethylsulfate, and then (b) adding an alkali or alkaline earth metal hydroxide at a temperature from about 25° C. to about −15° C.

(R)—N-benzyl-2-(benzyloxycarbonylamino)-3-hydroxypropionamide of formula I can be prepared, for example, according to the process described in example 21 herein. The (R)—N-benzyl-2-(benzyloxycarbonylamino)-3-hydroxypropionamide of formula I can be purified, for example, by crystallizations as reported in examples 11-19 herein, prior to reacting it according to the process of the invention.

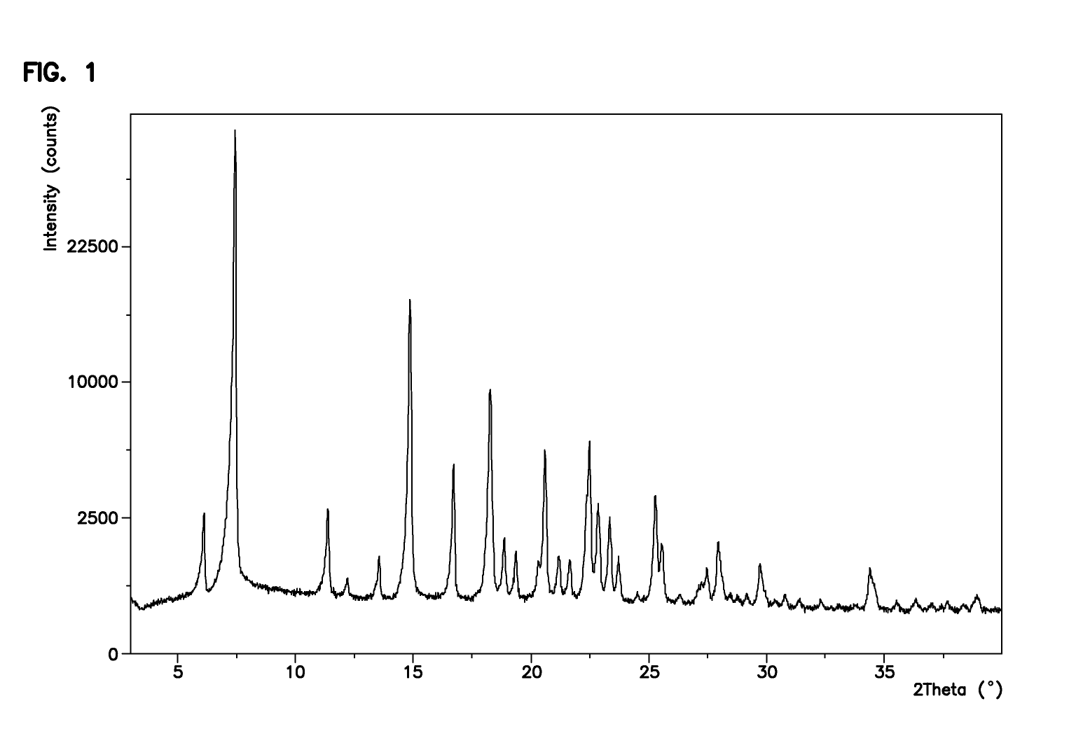

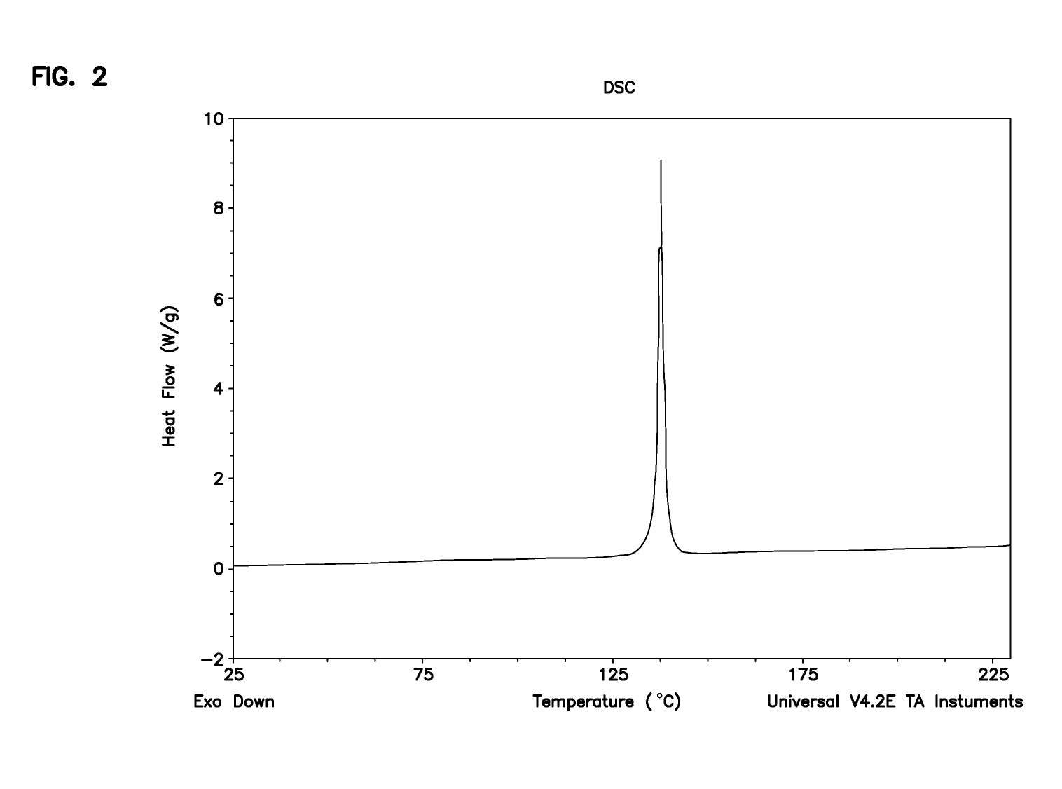

The obtained crystalline form of (R)—N-benzyl-2-(benzyloxycarbonylamino)-3-hydroxypropionamide is characterized by data selected from a group consisting of a PXRD pattern having peaks at about 7.4, 14.9, 16.7, 18.3, 20.6 and 22.5±0.2 degrees two-theta, a PXRD pattern described in FIG. 1 and combinations thereof. The crystalline form can be further characterized by a DSC thermogram described in FIG. 2. In some embodiments, the above crystalline of R)—N-benzyl-2-(benzyloxycarbonylamino)-3-hydroxypropionamide of formula I can be used to prepare (R)—N-benzyl-2-(benzyloxycarbonylamino)-3-methoxypropionamide of formula II.

According to some embodiments of the invention, (R)—N-benzyl-2-(benzyloxycarbonylamino)-3-hydroxypropionamide of formula I is combined with dimethylsulfate and a suitable solvent, such as a non-alcoholic solvent, to provide a suspension. The suspension can then be cooled prior the addition of the alkali or alkaline earth metal hydroxide.

The dimethylsulfate can be used in an amount from about 0.9 mole equivalent to about 100 mole equivalents, or from about 0.9 to about 20 mole equivalents based on the amount of the substrate, (R)—N-benzyl-2-(benzyloxycarbonylamino)-3-hydroxypropionamide of formula I.

Suitable non alcoholic solvents include for example, ketone solvents, ether solvents, halogenated hydrocarbon solvents, aromatic hydrocarbon solvents and mixtures thereof. Suitable ketone solvents include, for example, C₃-C₆ ketones such as acetone and methylethyl ketone. Suitable ether solvents include, for example C₄-C₇ ethers such as methyl-t-butyl ether, dioxane and tetrahydrofuran. Suitable halogenated hydrocarbon solvents include, for example, C₁-C₄ halogenated hydrocarbons such as methylene chloride, dichloroethane and trichloroethane. Suitable aromatic hydrocarbons include, for example, C₆-C₉ aromatic hydrocarbon, such as toluene, xylene and mesitylene.

The addition of the alkali or alkaline earth metal hydroxide can be done at a temperature from about 20° C. to about −15° C., or from about 0° C. to about −10° C., or from about 5° C. to about 0° C.

The alkali metal hydroxide can be for example sodium hydroxide, potassium hydroxide, lithium hydroxide. Examples of alkaline earth metal hydroxides include magnesium hydroxide and calcium hydroxide.

Typically the alkali metal or alkaline earth metal hydroxide is in an aqueous solution. The aqueous solution can be at any suitable concentration, for example, at a concentration from about 2% (w/w) to about saturation, or from about 2% to about 50%, or from about 30% to about 50% (w/w). The alkali metal or alkaline earth metal hydroxide is used in a molar ratio from about 0.4 to about 1.1 mole equivalents of the alkali metal or alkaline earth metal hydroxide per equivalent of dimethyl sulfate.

The addition of the alkali or alkaline earth metal hydroxide provides a reaction mixture in which (R)—N-benzyl-2-(benzyloxycarbonylamino)-3-methoxypropionamide of formula II is formed.

The addition of the alkali or alkaline earth metal hydroxide can be done in one portion.

Alternatively, to aid in maintaining the reaction temperature, the addition of the alkali or alkaline earth metal hydroxide can be done over a time interval, either portion-wise or dropwise or in a steady stream. For example the addition can be done over a period of about 5 minutes to about 12 hours, or from about 30 minutes to 6 hours or from 30 minutes 2 hours.

The above process can optionally be conducted in the presence of a suitable phase transfer catalyst (PTC). Suitable PTC's include for example quaternary ammonium, phosphonium or sulfonium salts.

Suitable quaternary ammonium salts include for example tetrabutylammonium bromide, tributylmethylammonium chloride and tetrabutylammonium sulfate. The reaction is maintained for a sufficient time to form (R)—N-benzyl-2-(benzyloxycarbonylamino)-3-methoxypropionamide of formula II. Typically, the reaction is maintained for a period from about 3 to about 10 hours.

The product, (R)—N-benzyl-2-(benzyloxycarbonylamino)-3-methoxypropionamide of formula II, can be recovered from the reaction mixture, by precipitating it. According to some embodiments, the reaction mixture can be a two phase reaction mixture. In such instances, the precipitation can be done, for example, by separating the organic phase, concentrating it, and optionally, further cooling it to obtain a suspension. The concentrated organic phase can be cooled to a temperature of about 5° C. to about 0° C., for a period from about 2 to about 6 hours, or about 4 hours to facilitate precipitation.

The (R)—N-benzyl-2-(benzyloxycarbonylamino)-3-methoxypropionamide of formula II produced by the process of the invention has a high level of purity. It can have a total purity of at least 96% area by HPLC, According to some embodiments, it can have a purity of at least 97% area by HPLC.

In some embodiments, the obtained (R)—N-benzyl-2-(benzyloxycarbonylamino)-3-methoxypropionamide of formula II can be purified, for example, by crystallization as reported in examples 5-10. Typically, the purified (R)—N-benzyl-2-(benzyloxy carbonylamino)-3-methoxypropionamide of formula II as obtained by crystallization is crystalline.

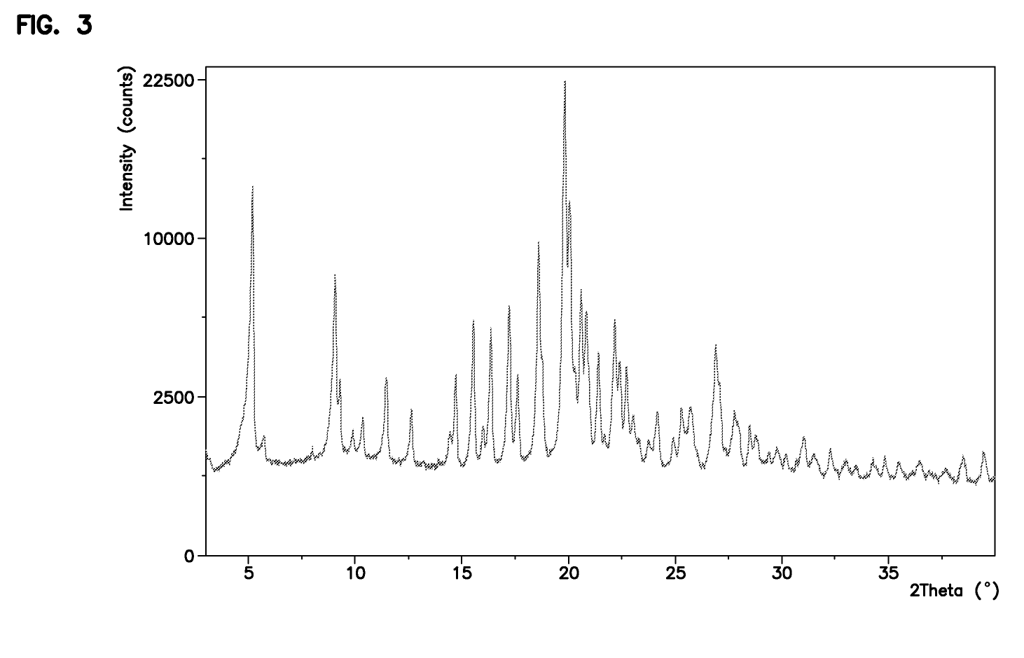

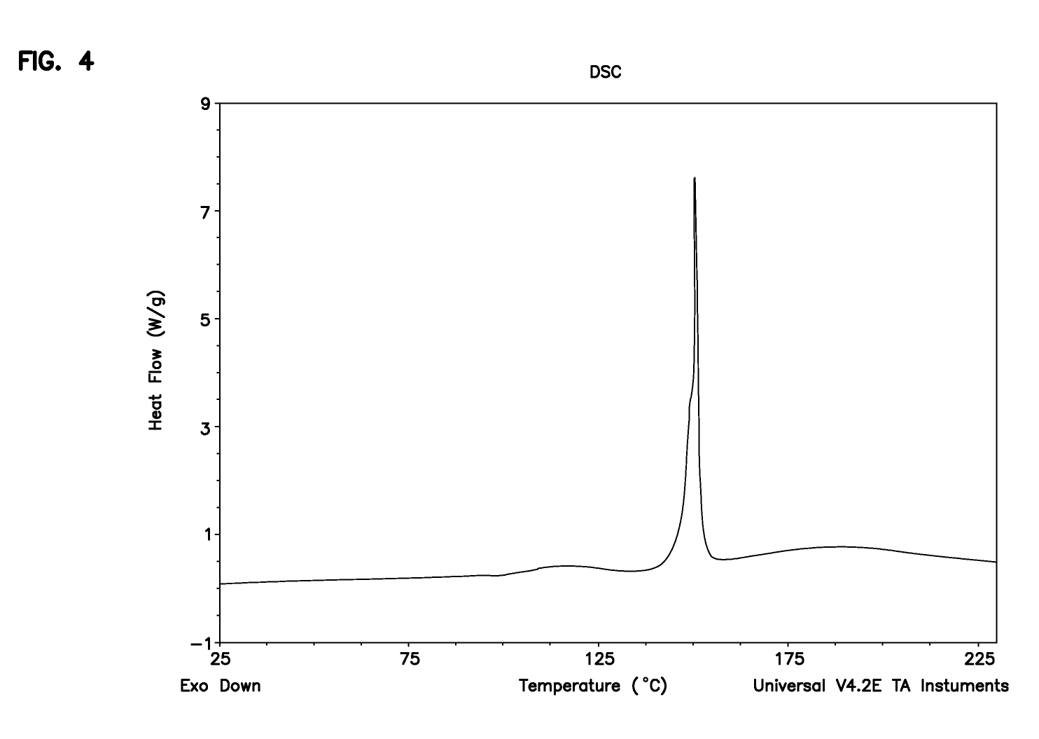

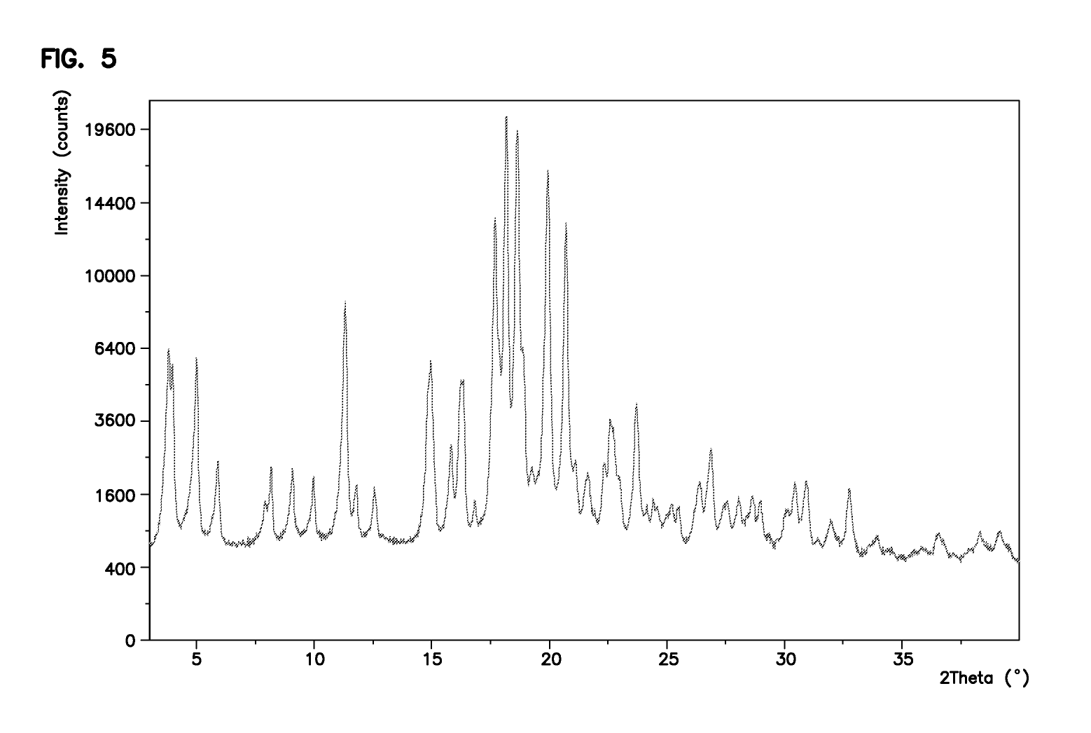

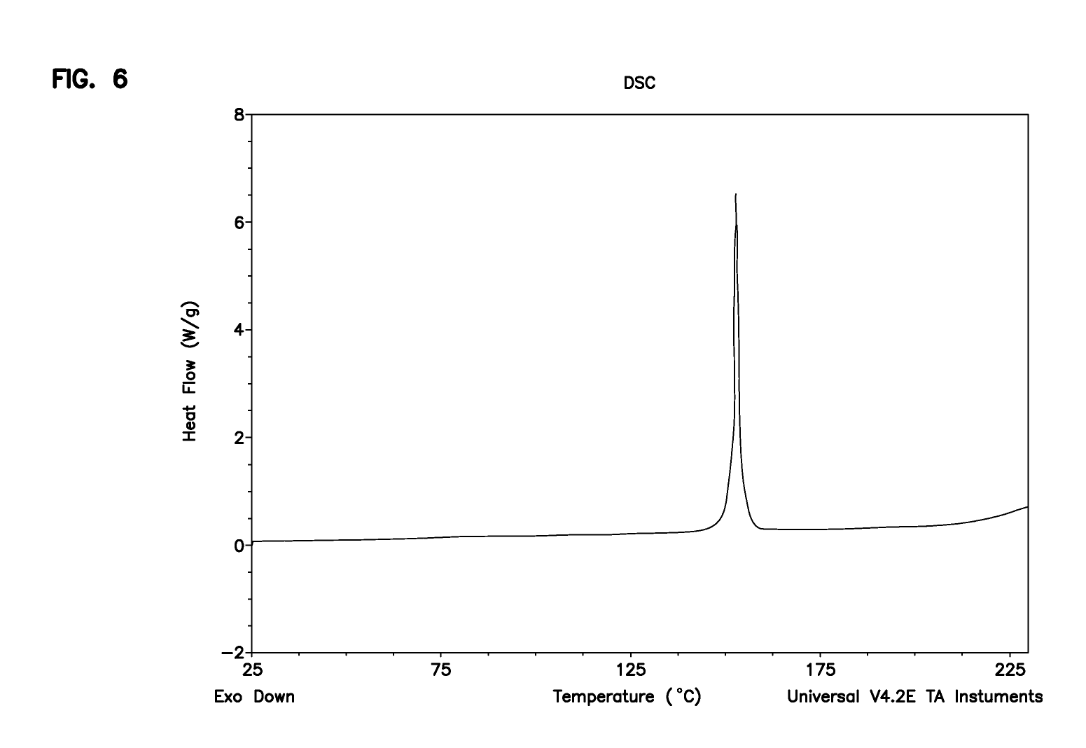

Reported herein are two crystalline forms of (R)—N-benzyl-2-(benzyloxycarbonylamino)-3-methoxypropionamide of formula II. The first crystalline form of (R)—N-benzyl-2-(benzyloxycarbonylamino)-3-methoxypropionamide, designated form I, is characterized by data selected from a PXRD pattern having peaks at about 5.2, 9.1, 15.6, 17.2, 18.6, 19.8 and 20.1±0.2 degrees two-theta, a PXRD pattern described in FIG. 3 and combinations thereof. Form I can be further characterized by a DSC thermogram described in FIG. 4. The second crystalline form of (R)—N-benzyl-2-(benzyloxycarbonylamino)-3-methoxypropionamide, designated form II, is characterized by data selected from a PXRD pattern having peaks at about 3.8, 11.3, 15.0, 16.2, 17.7, 18.2 and 20.7±0.2 degrees two-theta, a PXRD pattern described in FIG. 5 and combinations thereof. Form II can be further characterized by a DSC thermogram as described in FIG. 6.

The obtained (R)—N-benzyl-2-(benzyloxycarbonylamino)-3-methoxypropionamide of formula II can be converted to Lacosamide.

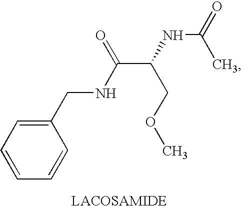

without the need of further purification; however the purified (R)—N-benzyl-2-(benzyloxycarbonylamino)-3-methoxypropionamide of formula II can also be used.

The conversion of the Formula II compound to Lacosamide can be done, for example, according to the process disclosed in example 3. As shown by example 3 it is evident that there is no need to purify Lacosamide produced by the process of the invention in order to obtain it in high quality. Further, the Lacosamide is obtained by the process of the invention in a good yield from about 60% to about 90%, or of at least 70%, calculated per the mole of (R)—N-benzyl-2-(benzyloxycarbonylamino)-3-methoxypropionamide of formula II.

Lacosamide that is obtained from such high quality of (R)—N-benzyl-2-(benzyloxycarbonylamino)-3-methoxypropionamide of formula II according to the process of the invention is also very pure, for example having a purity of at least 98%, or at least 99% or at least 99.3% area by HPLC. In addition, the N-Me Lacosamide impurity is not detected by HPLC in the product of the process of the invention.

### EXAMPLES

### A. Analytical Methods

### HPLC Purity of (R)—N-benzyl-2-(benzyloxycarbonylamino)-3-methoxypropionamide of Formula II and Lacosamide

An instrument used for the analysis was Agilent 1200 with an octadecylsilyl silica gel column (Phenomenex Inertsil ODS-3, 100 Å, 150 mm×4.6 mm, 5 μm) installed and thermostated at 35° C. The instrument was equipped with a DAD detector with wavelength set to 210 nm (BW 4 nm, ref. 360 nm, BW 100 nm).

The flow rate was set at 1.0 ml/min and the injection volume was set to 10 μl, with wash-vial function on. The wash vial was filled with the mixture of water and ACN (50/50, v/v). The autosampler temperature was set to 25° C.

Analytical runs were performed with gradient elution. Mobile phase “A” was water:ACN+0.1% TFA=8:2 and mobile phase “B” was ACN+0.1% TFA.

### Gradient:

| | % of mobile phase | % of mobile phase |
|---|---|---|
| Time | “A” | “B” |
| 0.00 | 100 | 0 |
| 2.00 | 100 | 0 |
| 26.00 | 67 | 33 |
| 29.00 | 67 | 33 |
| 32.00 | 100 | 0 |

The analytical run-time was 32 minutes.

Samples were accurately weighed and dissolved in diluent (mixture of water and ACN+0.1% TFA (50/50, v/v)) in order to obtain a test solution having a known concentration of about 1.0 mg/ml.

### B. HPLC Optical Purity of (R)—N-benzyl-2-(benzyloxycarbonylamino)-3-methoxypropionamide of Formula II

An instrument used for the analysis was Agilent 1200 with a-amylose tris (3,5-dimethylphenylcarbamate) coated on silica gel—column (Daicel Chiralpak AD, 250 mm×4.6 mm, 10 μm) installed and thermostated at 35° C. Instrument had VWD detector with wavelength set to 210 nm.

The flow rate was at 1.0 ml/min and the injection volume was set to 25 IA, with wash-vial function on. The wash vial was filled with diluent. The autosampler was at ambient temperature. Analytical runs were performed with isocratic elution using n-hexane: 2-propanol (90:10, v/v) as the mobile phase. The analytical run-time was 30 minutes. Samples were accurately weighed and dissolved in diluent (mixture of n-hexane and 2-propanol (80/20, (v/v)) in order to obtain a test solution having known concentration of about 1.0 mg/ml.

### C. HPLC Optical Purity

### Lacosamide

An instrument used for the analysis was Agilent 1200 with a—cellulose tris (3,5-dimethylphenylcarbamate) coated on silica gel—column (Daicel Chiralcel OD-H, 250 mm×4.6 mm, 5 μm) installed and thermostated at 35° C. The instrument was equipped with a VWD detector with wavelength set to 210 nm. The flow rate was set at 1.0 ml/min and the injection volume was set to 10 μl, with wash-vial function on. The wash vial was filled with diluent. The autosampler was at ambient temperature.

Analytical runs were performed with isocratic elution using n-hexane:2-propanol (85:15, v/v) as mobile phase. The analytical run-time was 30 minutes.

Samples were accurately weighed and dissolved in diluent (mixture of n-hexane and 2-propanol (80/20, (v/v)) in order to obtain a test solution having known concentration of about 1.0 mg/ml.

### D. Powder X-Ray Diffraction (PXRD) Analysis

After being powdered using a mortar and pestle, the samples were applied directly on silicon plate holder. The X-ray powder diffraction pattern was measured with Philips X'Pert PRO X-ray powder diffractometer, equipped with Cu irradiation source=1.54184 \acute{Å} (\acute{Å}ngstrom), X'Celerator (2.022° 2Θ) detector. Scanning parameters: angle range: 3-40 deg., step size 0.0167, time per step 50 s or 100 s, continuous scan. The accuracy of peak positions was defined as ±0.2 degrees due to experimental differences like instrumentations and sample preparations.

### E. Differential Scanning Calorimetry (DSC)

DSC analysis was performed on Q 1000 MDSC TA instruments with heating rate of 10° C./min, under nitrogen flow of 50 ml/min, hermetic aluminum, closed pan (with hole) was used, sample mass was about 1-5 mg.

### Example 1

### Preparation of (R)—N-benzyl-2-(benzyloxycarbonylamino)-3-methoxypropionamide

To a three-neck flask equipped with a mechanical stirrer, dropping funnel and thermometer (R)—N-benzyl-2-(benzyloxycarbonylamino)-3-hydroxypropionamide (5.0 g, 15.2 mmol), acetone (67 ml) and dimethyl sulfate (10.5 ml, 110.7 mmol) were added. The resulting suspension was cooled to 0-5° C. and an aqueous solution of sodium hydroxide (30%, 9.4 ml, 93.8 mmol) was added dropwise over a time interval of 2 hours while maintaining the temperature at 0-5° C. The resulting reaction mixture was stirred for 4 hours at 0-5° C. The reaction mixture was observed to be a two-phase system. The layers were separated and the acetone layer was concentrated to half of the starting volume, stirred at 0-5° C. for an hour. The product (R)—N-benzyl-2-(benzyloxycarbonylamino)-3-methoxypropionamide was then isolated by filtration (4.0 g, 11.7 mmol in 2 crops, 77% Yield); HPLC purity 97%, ee>99.8%.

### Example 2

### Preparation of R)—N-benzyl-2-(benzyloxycarbonylamino)-3-methoxypropionamide

To a three-neck flask equipped with a mechanical stirrer, dropping funnel and thermometer was added (R)—N-benzyl-2-benzyloxycarbonylamino)-3-hydroxypropionamide (5.0 g, 15.2 mmol), tetrahydrofuran (67 ml), dimethyl sulfate (10.5 ml, 110.7 mmol) and tetrabutylammonium bromide (0.20 g, 0.6 mmol). The resulting suspension was cooled to 0-5° C. and an aqueous solution of sodium hydroxide (50%, 2.81 ml, 53.7 mmol) was added dropwise over a time interval of 60 minutes while maintaining temperature at 0-5° C. The reaction mixture was stirred for 4 hours at 0-5° C. The reaction mixture was observed to be a two-phase system. The layers were separated and the acetone layer was concentrated to half of the starting volume, stirred at 0-5° C. for an hour. The product (R)—N-benzyl-2-(benzyloxycarbonylamino)-3-methoxypropionamide was then isolated by filtration. (4.0 g, 11.7 mmol in 1 crop, 77% Yield). HPLC purity of obtained product was 96%, ee>99.8%)

### Example 3

### Lacosamide Preparation

(R)—N-benzyl-2-(benzyloxycarbonylamino)-3-methoxypropionamide (1 g, 2.9 mmol), (prepared in example 1), was dissolved in ethyl acetate (50 ml). To this solution was added Pd/C 10% (0.25 g). The resulting mixture was hydrogenated for 1 hour at 2 bar pressure at 25° C. The reaction mixture was then filtered to remove the catalyst and the filtrate was concentrated on a rotary evaporator to 15 ml volume. To the concentrate was added triethylamine (0.45 ml, 3.2 mmol) and acetic anhydride (0.31 ml, 3.3 mmol), and the resulting mixture was stirred at room temperature for 1 hour. The product (R)-2-acetamido-N-benzyl-3-methoxypropionamide was isolated from ethyl acetate/heptane (1:1) mixture at 0-5° C. (520 mg, 2.1 mmol, 72%, [α]_{D}+15.5°; HPLC purity 99.3%; ee>99.8%)

### Example 3a

### Lacosamide Preparation

(R)—N-benzyl-2-(benzyloxycarbonylamino)-3-methoxypropionamide (167 g, 458 mmol), (prepared as in example 1), was dissolved in ethyl acetate (1700 ml). To this solution was added Pd/C 10% (17 g). The resulting mixture was hydrogenated for 1 hour at 5-6 bar pressure at 30-35° C. The reaction mixture was then filtered to remove the catalyst. To the filtrate was added triethylamine (89.5 ml, 642 mmol) and acetic anhydride (51.9 ml, 550 mmol) in a dropwise manner. The resulting reaction mixture was stirred at room temperature for 1 hour. The product (R)-2-acetamido-N-benzyl-3-methoxypropionamide was isolated from ethyl acetate/n-heptane (1:1) mixture at 0-5° C. (98.7 g, 394.3 mmol, 86% Yields, HPLC purity 99.2 area %; ee>99.8%).

### Example 4

### Comparative Example

### Preparation of (R)—N-benzyl-2-(benzyloxycarbonylamino)-3-methoxypropionamide Using Silver Oxide and Methyl Iodide

To a roundbottom flask, protected from light, (R)—N-benzyl-2-(benzyloxycarbonylamino)-3-hydroxypropionamide (1.0 g, 3.04 mmol), acetonitrile (211 ml) and silver oxide (4.89 g, 21.1 mmol) were added. The resulting suspension was mixed for 30 minutes and methyl iodide (2.61 ml, 40.6 mmol) was added. The resulting reaction mixture was stirred at room temperature for 4 days. The reaction mixture was then filtered through diatomaceous earth to produce a gray solution. Small particles of silver oxide were observed in the filtrate, but these were removed by repeating the filtration,

The resulting solution was evaporated in vacuo to obtain grey crystals of (R)—N-benzyl-2-(benzyloxycarbonylamino)-3-methoxypropionamide (0.99 g, 2.42 mmol, 95% yield). HPLC purity 93.9%. The crystals were purified using column chromatography with methanol:chloroform 1:19. The collected fractions of intermediate II were evaporated to dryness to provide the purified product (4.94 g; 94%, assay 87.8%; HPLC purity 90.2 area %; N-Me intermediate II 6.7 area %

### Example 4a

### Comparative Example

### Preparation of Lacosamide Via the Intermediate which was Prepared According to Example 4

(R)—N-benzyl-2-(benzyloxycarbonylamino)-3-methoxypropionamide (4 g, 11.7 mmol), (prepared in example 4), was dissolved in ethyl acetate (50 ml). To this solution was added Pd/C 10% (0.5 g). The resulting mixture was hydrogenated for 5.5 hours at 5 bar pressure at 30-35° C. The reaction mixture was then filtrated to separate the catalyst. To the filtrate was added triethylamine (2.31 ml, 16.6 mmol) and acetic anhydride (1.35 ml, 14.3 mmol) and the resulting reaction mixture was stirred at room temperature for 1 hour. The product (R)-2-acetamido-N-benzyl-3-methoxypropionamide was isolated from ethyl acetate/heptane (1:1) mixture at 0-5° C. (1.47 mg, 5.9 mmol, 50.3%, [α]_{D}+15.5°; HPLC purity 99.4%; ee>99.8%). Mother liquor was evaporated till dryness (2.13 g, HPLC purity Lacosamide 69.4 area %, N-Me Lacosamide 20.0 area %; assay 67%).

### Example 5

### Preparation of Crystalline Form I of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 1.0 ml of acetonitrile by heating in a small flask. The resulting solution in a closed flask was left to cool down to room temperature. After about 1 day white crystals formed and were separated by filtration. The obtained product was analyzed by powder X-ray diffraction and DSC. The resulting XRPD pattern is shown in FIG. 3 and the resulting DSC thermogram as shown in FIG. 4.

### Example 6

### Preparation of Crystalline Form II of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 0.6 ml of methyl ethyl ketone by heating in small flask. The resulting solution in a closed flask was left to cool down to room temperature. After about 1 day white crystals formed and were separated by filtration.

### Example 7

### Preparation of Crystalline Form II of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 0.8 ml of methyl isobutyl ketone by heating in small flask. The resulting solution in a closed flask was left to cool down to room temperature. After about 1 day white crystals formed and were filtered. The obtained crystals were analyzed by PXRD and DSC. The PXRD pattern is shown in FIG. 5, and the DSC thermogram as shown in FIG. 6.

### Example 8

### Preparation of Crystalline Form II of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 0.6 ml of 1-propanol by heating in small flask. The solution in a closed flask was left to cool down to room temperature. After about 1 day white crystals formed and were filtered.

### Example 9

### Preparation of Crystalline Form II of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 0.8 ml of 2-propanol by heating in small flask. The solution in a closed flask was left to cool down to room temperature. After about 1 day white crystals formed and were filtered.

### Example 10

### Preparation of Crystalline Form II of (R)—N-benzyl-2-(benzyloxycarbonylamino)-3-methoxypropionamide

(R)—N-benzyl-2-(benzyloxycarbonylamino)-3-methoxypropionamide (30 mg) was dissolved in 1.4 ml of toluene by heating in small flask. The solution in a closed flask was left to cool down to room temperature. After about 1 day white crystals formed and were filtered.

### Example 11

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of acetone by heating in small flask. The solution in a closed flask was left to cool down to room temperature. After about 1 day white crystals formed and were filtered.

### Example 12

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of acetonitrile by heating in small flask. The solution in a closed flask was left to cool down to room temperature. After about 1 day white crystals formed and were filtered.

### Example 13

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of ethanol by heating in small flask. The solution in a closed flask was left to cool down to room temperature. After about 1 day white crystals formed and were filtered.

### Example 14

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of ethyl acetate by heating in small flask. Solution in a closed flask was left to cool down to room temperature. After about 1 day white crystals were filtered.

### Example 15

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.4 ml of methanol by heating in small flask. Solution in a closed flask was left to cool down to room temperature. After about 1 day white crystals were filtered.

### Example 16

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of methyl ethyl ketone by heating in small flask. Solution in a closed flask was left to cool down to room temperature. After about 1 day white crystals were filtered. Obtained crystals have an XRPD pattern as shown in FIG. 1 and a DSC thermogram as shown in FIG. 2.

### Example 17

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of methyl isobutyl ketone by heating in small flask. Solution in a closed flask was left to cool down to room temperature. After about 1 day white crystals were filtered.

### Example 18

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of 1-propanol by heating in small flask. Solution in a closed flask was left to cool down to room temperature. After about 1 day white crystals were filtered.

### Example 19

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.6 ml of 2-propanol by heating in small flask. Solution in a closed flask was left to cool down to room temperature. After about 1 day white crystals were filtered.

### Example 20

### Preparation of Crystalline Form of (R)—N-benzyl-2-(benzyloxycarbonylamino)-3-hydroxypropionamide

(R)—N-benzyl-2-(benzyloxycarbonylamino)-3-hydroxypropionamide (30 mg) was dissolved in 0.8 ml of toluene by heating in small flask. Solution in a closed flask was left to cool down to room temperature. After about 1 day white crystals were filtered.

### Example 21

### Preparation of (R)—N-benzyl-2-(benzyloxycarbonylamino)-3-hydroxypropionamide

To a three-neck flask equipped with a mechanical stirrer, dropping funnel and thermometer was added (R)-2-(benzyloxycarbonylamino)-3-hydroxypropanoic acid (20 g, 83.6 mmol) and ethyl acetate (700 ml). The reaction mixture was cooled down to about −10° C. Next, 4-methylmorpholine (9.6 ml, 87.0 mmol) was added and after 5 minutes of stirring, isobutyl chloroformate (11.4 ml, 87.2 mmol) was added. To the thus-formed suspension after 5 minutes benzylamine (9.6 ml, 88.0 mmol) was added in one portion. Vigorous stirring was continued for another 10 min and the cooling bath was removed and the reaction mixture was allowed to warm to RT (20-23° C.) within 60-75 min. Stirring was continued for 1 hour. Next the suspension was warmed up to about 28-30° C. and dissolved by the addition of 1% aqueous HCl (140 ml) that had been pre-warmed to about 30° C. After stirring for a few minutes at 28-30° C. the clear reaction solution was transferred into a reparatory funnel and the acidic aqueous layer was separated. The organic layer was washed with warm (35° C.) distilled water (4×120 ml). The organic layer was then washed with warm (35° C.) saturated NaCl solution (120 ml). The clear organic layer was then transferred into a three neck roundbottom flask and was stirred with anhydrous Na₂SO₄ (30 g) at temperature 33-37° C. (Note 4) for 30 min. The sodium sulfate was then removed by filtration and washed with ethyl acetate (8 ml). The combined filtrate was partially concentrated under reduced pressure to 390-400 g. The partially concentrated warm solution (with small amounts of precipitate) was transferred into a reactor and was warmed to 35-40° C., and n-heptane (200 ml) was added dropwise over a period of 30-45 min. The resulting thick suspension was stirred without heating for 30 min and then was cooled to 5° C. and stirred for another 30 min. The product was separated by filtration, washed, first with a cold mixture (5° C.) ethyl acetate/n-heptane 1:1 (50 ml) and then with heptane (50 ml). The product was dried for 6 hours at 60° C. Yield: 22.25 g (81%).

### Example 22

### Preparation of R)—N-benzyl-2-(benzyloxycarbonylamino)-3-methoxypropionamide

### Comparative Example

To a reaction flask was added (R)—N-benzyl-2-(benzyloxycarbonylamino)-3-hydroxypropionamide (13.73 g; 41.8 mmol), toluene (43 ml) and tetrabutylammonium bromide (0.5 g). The resulting very thick, immiscible suspension was diluted with toluene (37 ml) and then cooled below 10° C. To this cooled mixture was added a previously prepared solution of sodium hydroxide (1.67 g of NaOH in 6.5 ml of distilled water) was added. The resulting suspension was stirred for 30 minutes at 5-10° C. and dimethyl sulfate (15.8 ml) was added. To the resulting mixture was added NaOH (50% solution: 7.5 g of NaOH in 7.5 ml of distilled water). The resulting suspension was stirred for 60 minutes at 5-10° C. and water (26 ml) was added to form a two phase mixture. The layers were separated. The pH of the water layer was adjusted to 3.4 with citric acid (50% solution in water). The resulting mixture was extracted with methylene chloride (2×35 and 1×27 ml). The methylene chloride extracts were combined and evaporated to dryness to provide 40 mg of crystalline product. Toluene layer was analyzed by HPLC. Analysis determined that the product consisted of 40% of the N-methylated by product, 24 area % of (R)—N-benzyl-2-(benzyloxycarbonylamino)-3-methoxypropionamide was formed and 32.5 area % was unreacted (R)—N-benzyl-2-(benzyloxycarbonylamino)-3-hydroxypropionamide.

What is claimed is:

1. A process for preparing (R)—N-benzyl-2-(benzyloxycarbonylamino)-3-methoxypropionamide according to formula II: 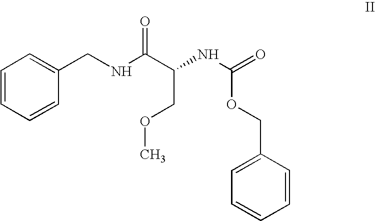
said process comprising
(a) combining (R)—N-benzyl-2-(benzyloxycarbonylamino)-3-hydroxypropionamide according to formula I: 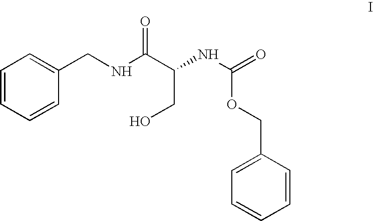
and dimethylsulfate to form a mixture;
(b) adding to the mixture an alkali or alkaline earth metal hydroxide at a temperature of about 25° C. to about −15° C. to form a reaction mixture; and
(c) optionally isolating (R)—N-benzyl-2-(benzyloxycarbonylamino)-3-methoxypropionamide from the reaction mixture.

2. The process of claim 1, wherein step (a) comprises combining the (R)—N-benzyl-2-(benzyloxycarbonylamino)-3-hydroxypropionamide of formula I with dimethylsulfate and a non-alcoholic solvent, forming a suspension.

3. The process of claim 2, further comprising cooling the suspension prior to step (b).

4. The process of claim 2, wherein the non alcoholic solvent is a ketone solvent, an ether solvent, a halogenated hydrocarbon solvent, an aromatic hydrocarbon solvent or a mixture thereof.

5. The process of claim 1, wherein the alkali metal or alkaline earth metal hydroxide is used in a molar ratio from about 0.4 to about 1.1 mole equivalents per mole equivalent of dimethyl sulfate.

6. The process of claim 1, wherein the product (R)—N-benzyl-2-(benzyloxycarbonylamino)-3-methoxypropionamide of formula II has a total purity of at least 96% area by HPLC.

7. A process for preparing lacosamide, comprising preparing (R)—N-benzyl-2-(benzyloxycarbonylamino)-3-methoxypropionamide according to claim 1, and converting the (R)—N-benzyl-2-(benzyloxycarbonylamino)-3-methoxypropionamide to lacosamide.
